(19) United States
(12) Patent Application Publication
(10) Pub. No.: US 2019/0110735 A1
(43) Pub. Date: Apr. 18, 2019

(54) Methods and Systems for Assessment of Cutaneous Autonomic Nerve Function

(71) Applicant: O'Brien Medical, Orono, ME (US)

(72) Inventor: Todd O'Brien, Orono, ME (US)

(21) Appl. No.: 16/160,729

(22) Filed: Oct. 15, 2018

Related U.S. Application Data
(60) Provisional application No. 62/572,269, filed on Oct. 13, 2017.

(51) Int. Cl. A61B 5/00; A61B 5/0295; A61B 5/026

(52) U.S. Cl. CPC A61B 5/4035; A61B 5/0295; A61B 5/0022; A61B 5/0051; A61B 2562/0238; A61B 5/0261; A61B 5/7246; A61B 5/7278; A61B 5/742

(57) ABSTRACT

A system and method for the non-invasive assessment of cutaneous autonomic nerve function through the application of external vibrational stimulus is disclosed. System embodiments comprising an electronic vibration source and photoplethysmographic (PPG) sensor enable measurement of vasomotor responses to vibrational stimuli. This response, facilitated by Pacinian channel-mediated stimulation of small fiber autonomic nerves, is quantified by PPG waveform analysis of transient skin vasoconstriction. Utilizing this normally elicited vasoconstriction reflex as the reference standard, small fiber autonomic nerve deficits revealed by absent or diminished vasomotor responses can be detected. Measurement of these deficits will enable rapid, non-invasive assessment of small fiber nerve degeneration in a variety of medical conditions including diabetes, hypothyroidism and chemotherapy-induced peripheral neuropathy.

### CROSS REFERENCE TO RELATED APPLICATIONS

This application claims the benefit of, and priority to U.S. Provisional Patent Application Ser. No. 62/572,269, filed Oct. 13, 2017, the content of which is incorporated herein by reference herein in its entirety.

### BACKGROUND OF THE DISCLOSURES

Small and large fiber neuropathies are seen in a variety of medical conditions. The most common diagnosis associated with neuropathy is diabetes. Diabetic peripheral neuropathy (DPN), also known as distal symmetrical polyneuropathy, will be diagnosed in at least 50% of diabetic patients. DPN is usually a mix of large and small nerve fiber deficits with small fiber degeneration thought to precede large fiber pathology. Although objective, quantitative methods for diagnosing large fiber neuropathy are available through nerve conduction velocity and electromyographic testing, there remains an acknowledged lack of viable methods to quantitatively diagnose small fiber damage.

Cutaneous small fiber nerves are known to provide afferent feedback regarding pain, temperature and light touch. They also provide efferent functionality through autonomic vasomotor and sweat gland control. These small fiber nerves undergo degeneration in numerous medical conditions. In addition to diabetes, this can occur in hypothyroidism, chemotherapy-induced neuropathy, Sjogren's syndrome, Lupus, vasculitis, sarcoidosis, nutritional deficiency, Celiac disease, Lyme disease, HIV, amyloidosis and alcoholism. Despite the prevalence of the problem, there are no widely-adopted, non-invasive, quantitative tests available to diagnose it. Instead, clinicians rely on subjective, psychophysical neurological assessments such as the pinprick test to elicit a pain response. Development of a reliable objective test is of critical importance especially in diabetic patients as small fiber neuropathy often results in loss of protective sensation leading to subsequent foot ulcers, infections and amputations.

Lacking accurate testing methods, several alternative approaches have recently been developed. Unfortunately, these methods are expensive, experimental, or invasive. These tests include counting the number of small nerve fibers though skin biopsies, measurement of sweat gland function, measurement of skin vasodilation in response to heat though laser Doppler imaging and measurement of evoked action potentials at the level of the brain through application of pain via laser heating of the skin. Among the newer experimental approaches is phase sensitive optical coherence reflectometry disclosed by Akkin (U.S. Pat. No. 9,326,719). This technique seeks to directly measure evoked neural potentials locally through assessment of nanometer scale skin surface displacements. Yet another experimental technique disclosed by Soliz (U.S. Pat. No. 8,868,157) teaches a method of assessing the vasoneurogenic response to thermal challenge on the skin of the foot through optical thermography.

In general, these methods have not become incorporated into clinical practice due to the above-noted shortcomings. The most popular of these tests is the skin biopsy which although accurate is invasive, resulting in a lower extremity wound. This consequence is seen by some clinicians as undesirable especially in diabetic patients predisposed to poor wound healing, infections and amputations. The current invention seeks to overcome the limitations of other small fiber nerve assessment methods by providing a rapid, non-invasive technique amenable to point-of-care testing and readily incorporated into established clinical care patterns.

### OBJECTS AND SUMMARY OF THE INVENTION

According to embodiments there is disclosed a method and system for detecting autonomic vasomotor responses to vibrational stimulus comprising, in combination;

At least a vibration source and photoplethysmographic (PPG) sensor controlled through electronic means wherein comparative analysis of waveform data acquired from baseline and post-stimulus measurements is automatically displayed on an interface for a user.

According to embodiments there is disclosed a method and system as denied within claim one, wherein the at least a vibrational source emits vibrations in the frequency range of from at least about 120 to approximately 130 Hz, and the like types ranges for different applications.

According to embodiments there is disclosed a system defined by of the instant disclosure, wherein said vibrational source emits vibrations of constant amplitude.

According to embodiments there is provided, a system wherein said vibrational source emits vibrations which are not of constant amplitude.

According to embodiments, there is a disclosed a syste, wherein the light emitting diodes within the PPG sensor emit light in the range of at least about 400 to approximately 900 nm.

According to embodiments there is disclosed a novel enhanced system as shown and described herein, whereby Pacinian Receptors signal small fiber autonomic nerves controlling skin-based vascular systems along with complementary and supplementary mechanisms related thereto and/or triggered thereby.

According to one embodiment of the present invention a method of measuring cutaneous autonomic nerve function is provided.

The patient contact member (#5 in drawing) is touched to the skin of the individual being tested. A PPG sensor embedded within the contact member is activated for a specified time to obtain a baseline waveform analysis.

This PPG sensor utilizes light emitting diodes (LED) to illuminate the skin and a photodiode to measure the light reflected back thereby providing a sensitive method of gauging changes in blood volume.

This first reading is assigned the value of 100% for each test. While maintaining contact with the skin, a standardized dose of vibrational stimulus is automatically provided for a specified time.

This vibrational stimulus is emitted at a constant amplitude in the frequency range between 120-130 Hz. This vibration causes vasoconstriction of skin capillaries and arterioles in normal tissue as experimentally demonstrated in numerous studies (see other references, 9-15).

The most widely accepted hypothesis to explain this phenomenon cites excitation of Pacinian corpuscles sensitive to vibration. These Pacinian receptors are then thought to signal small fiber autonomic nerves controlling skin vascular structures through a combination of local and/or centrally-mediated mechanisms.

At the end of the vibrational stimulus, a second PPG measurement is automatically taken. This second reading is then calculated as a percentage of the first reading and subtracted from that value. This result is then compared to normal reference values to provide an assessment of autonomic small fiber nerve function or dysfunction. For example, second readings close to 0% imply a diminished or absent vasoconstriction reflex indicative of severe small fiber neuropathy.

### BRIEF DESCRIPTION OF THE DRAWINGS

Various preferred embodiments are described herein with references to the drawings in which merely illustrative views are offered for consideration, whereby:

FIG. 1 is a schematic of a device according to the instant claims including user interface being a Display indicating percentage of blood volume reduction compared to baseline, inter alia. The identifiers for FIGS. 1, 2 and 3 are: 1. LCD Display, 2. Printed circuit board with microprocessor; 3; Control buttons, 4. Modular sub-units, 5. Patient contact member. 6. Vibrator, 7. Photodiode, 8 Light with emitting diodes, 9 is Patient Skin, 10. Power supply 10, is Light from LEDs and 11, Reflected light from tissue.

FIG. 2 is another schematic of a device according to the instant claims including user interface being a Display indicating percentage of blood volume reduction compared to. baseline, inter alia.

FIG. 3 is another schematic indicating steps according to the method disclosed in the instant application namely a plurality of schematics of a device according to the instant claims including user interfaces being a Display indicating percentage of blood volume reduction compared to baseline, inter alia.

### DETAILED DESCRIPTIONS

The present inventor has discovered that a device can be used to safely and accurately assess living tissue non-invasively. Namely, a graphic depiction of percentage of blood volume compared to a baseline is offered for consideration.

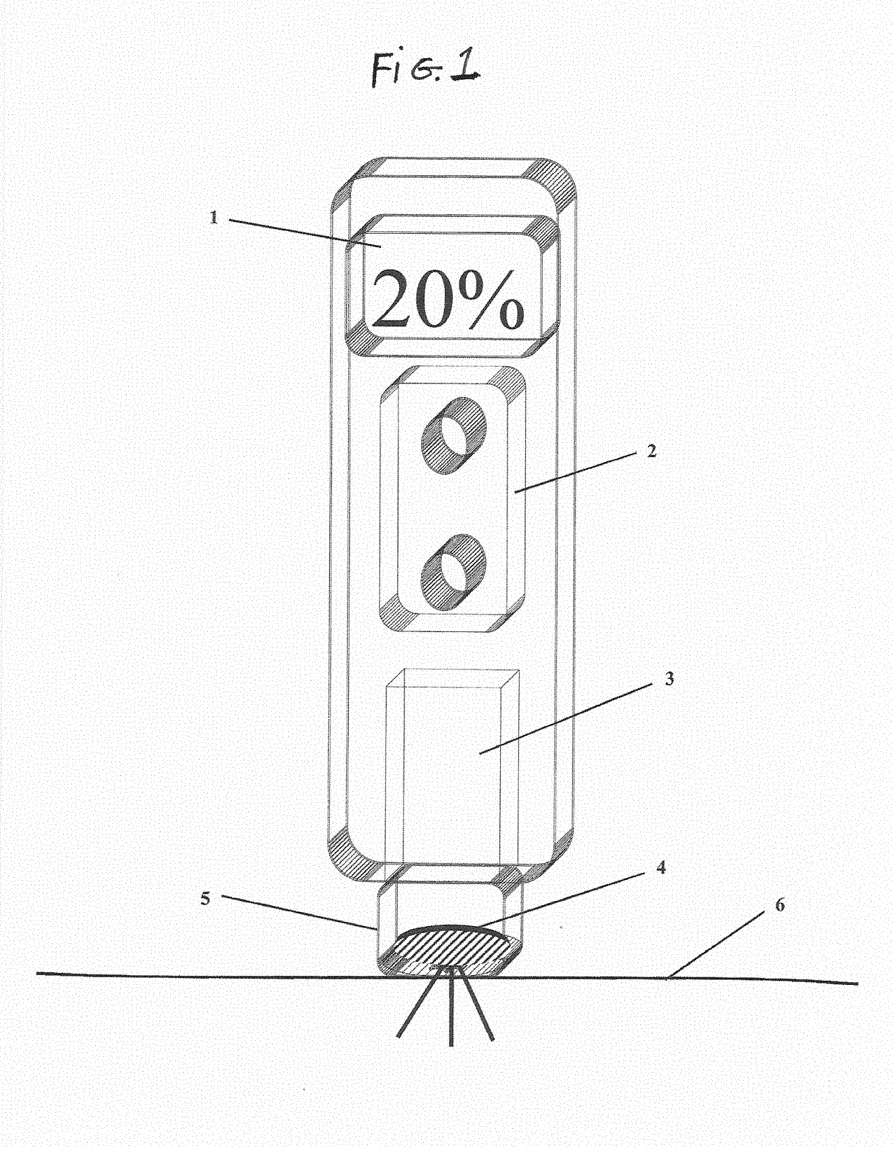

Those skilled in the art, referring to FIG. 1-3 can see how the invention works Turning now to FIG. 1, a device is shown, for example, with LCD Display 1, arrayed above printed circuit board 2 with on-board chip-set, microprocessor and wireless capability, and control buttons 3. Patient contact member 4, vibrator 5, and (not shown) photodiode, light emitting diodes and contact with patient skin 6.

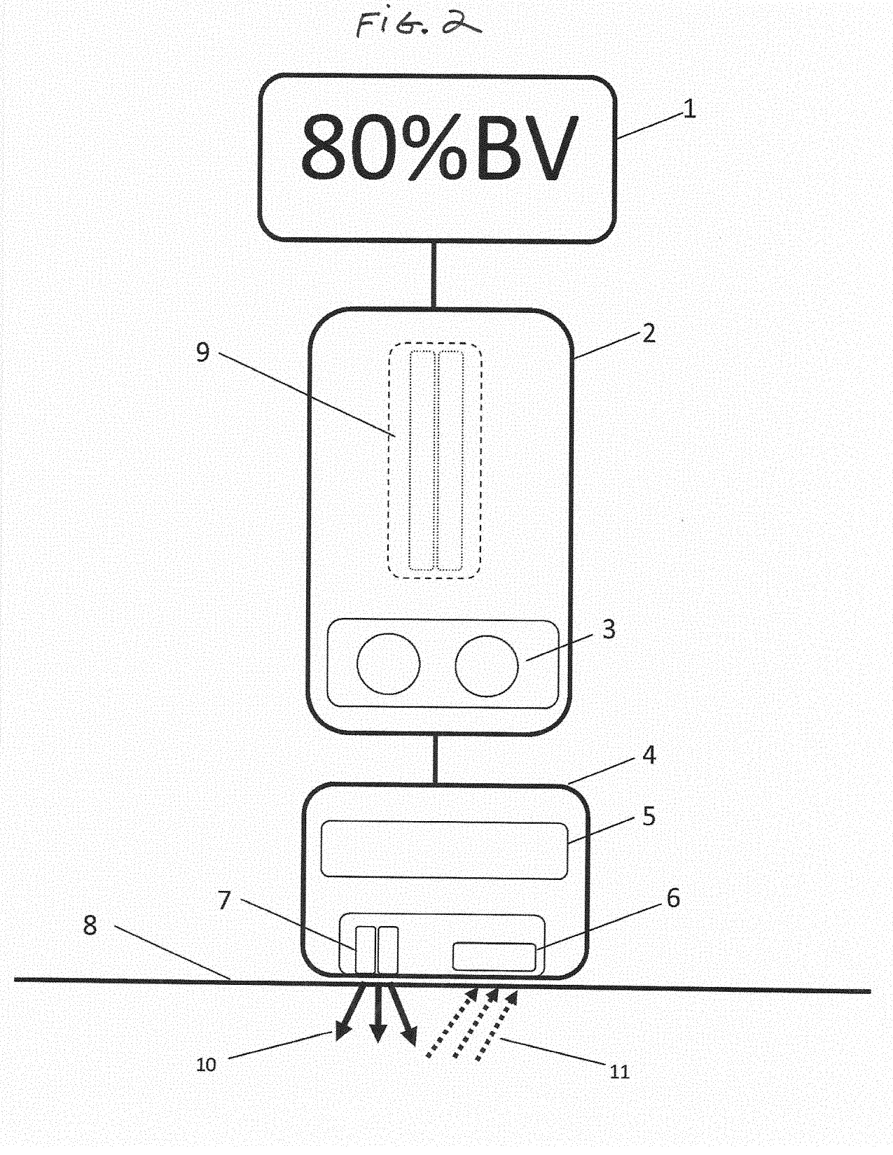

Turning now to FIG. 2, another schematic shows:

### Example

### Test Protocol

The user places the patient contact member on the skin.

The user starts the test through actuation of the appropriate controls.

The PPG sensor obtains the first reading of skin blood volume and waveform analysis is performed.

The vibration source is automatically activated providing vibrations to the skin following completion of PPG data acquisition.

The 2^{nd} PPG reading is automatically taken at the end of the vibration application period.

The percentage of skin blood volume reduction compared to baseline is displayed.

The test result can be compared to normal values to gauge autonomic small fiber nerve function.

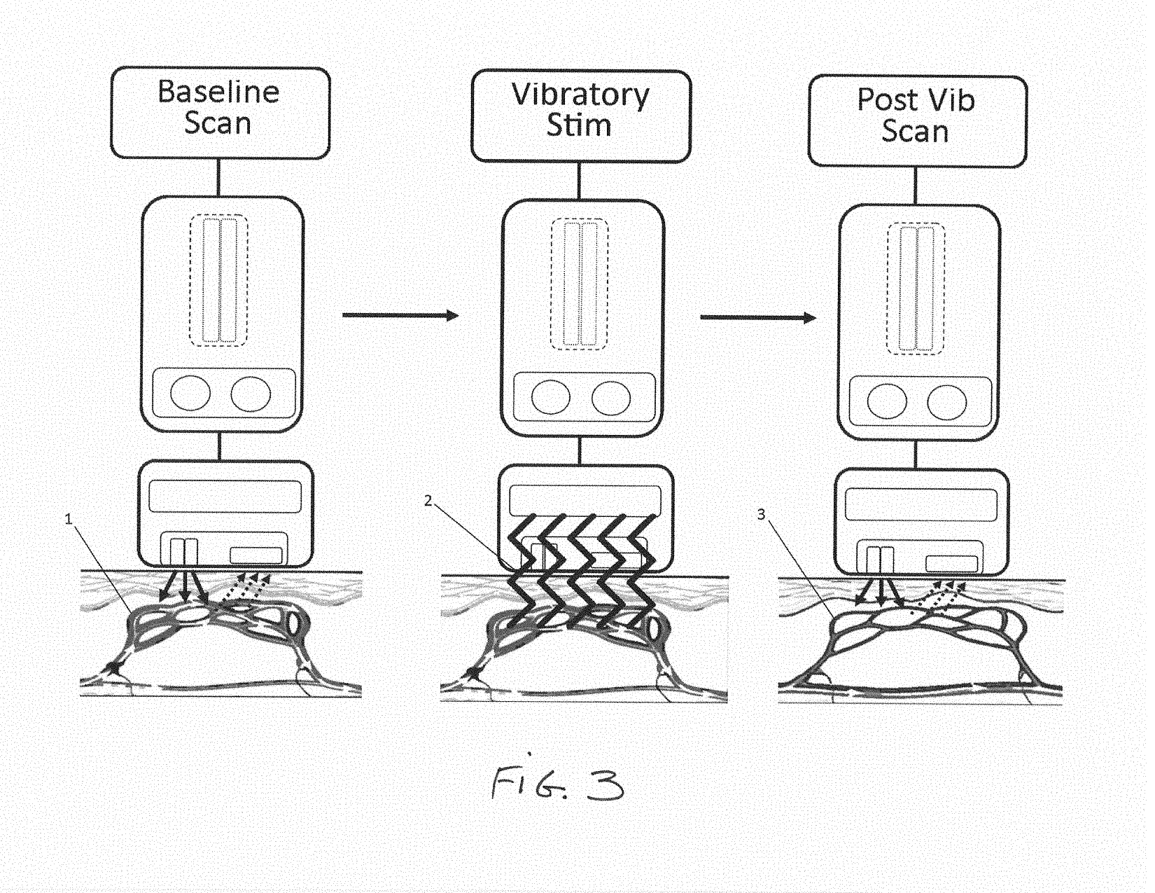

Turning now to FIG. 3, a process is shown whereby capillaries are shown in normal blood flow state as sensed by the present invention and displayed. Then, using vibration2, a vibratory skin reading is achieved. Capillaries having restricted flow 3 are sensed and the post vibrational scan provided showing small fiber damage by assessment of cutaneous autonomic nerve function. The identifiers for FIGS. 1, 2 and 3 are: 1. LCD Display, 2. Printed circuit board with microprocessor, 3; Control buttons, 4. Modular sub-units, 5. Patient contact member. 6. Vibrator, 7. Photodiode, 8 Light with emitting diodes, 9 is Patient Skin, 10 Power supply 10, is Light from LEDs and 11, Reflected light from tissue.

Corresponding reference characters generally are used to show corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. R is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent,, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language mans that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions, thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CAD), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### U.S. Patent Documents

U.S. Pat. No. 9,326,719 Akkin

U.S. Pat. No. 8,868,157 Soliz

U.S. Pat. No. 9,173,568 Gefen

U.S. Pat. No. 8,971,984 Freeman

U.S. Pat. No. 6,942,622 Turcott

US 2013/0131466 Wacogne

US 2016/0074661 Lipani

US 2006/0111652 McLeod

### OTHER REFERENCES

1. American Diabetes Association, Diabetes Care 2017 January; 40(Supplement 1): S88-S98. https://doi.org/10.2337/dc17-5013 accessed on Jun. 16, 2017.

2. Sharma S, Vas P R, Rayman G. Assessment of diabetic neuropathy using a point-of-care nerve conduction device shows significant associations with the LDIFLARE method and clinical neuropathy scoring. J Diabetes Sci Technol. 2015 January;9(1):123-31.

3. Krishnan, S. T. M., Rayman, G., “The LDIflare: A novel test of C-fiber function demonstrates early neuropathy in type 2 diabetes”, Diabetes Care, 2004, (27), v. 12, (2930-2935)

4. Di Stefano G I, La Cesa S, Leone C, Pepe A, Galosi E, Fiorelli M, Valeriani M, Lacerenza M, Pergolini M, Biasiotta A, Cruccu G, Truini A. Diagnostic accuracy of laser-evoked potentials in diabetic neuropathy. Pain. 2017 June;158(6):1100-1107.

5. Abuzinadah A R, Kluding P, Wright D, D'Silva L, Ryals J, Hendry B, Jawdat O, Herbelin L, McVey A L, Barohn R J, Dimachkie M M, Pasnoor M. Less is More in Diabetic Neuropathy Diagnosis: Comparison of Quantitative Sudomotor Axon Reflex and Skin Biopsy. J Clin Neuromuscul Dis. 2017 September;19(1):5-11.

6. Lauria G, Lombardi R. Small fiber neuropathy: is skin biopsy the holy grail? Curt Diab Rep. 2012 August;12(4):384-92. doi: 10.1007/s11892-012-0280-9.

7. Kasznicki J. Advances in the diagnosis and management o diabetic distal symmetric polyneuropathy. Arch

Med Sci. 2014 May 12;10(2):345-54.

8. Breiner A, Lovblom L E, Perkins B A, Bril V. Does the prevailing hypothesis that small-fiber dysfunction precedes large-fiber dysfunction apply to type 1 diabetic patients? Diabetes Care. 2014 May;37(5):1418-24.

9. Bovenzi M I, Lindsell C J, Griffin M I. Magnitude of acute exposures to vibration and finger circulation. Scand J Work Environ Health. 1999 June;25(3):278-84.

10. Ye Y, Griffin M J. Reductions in finger blood flow induced by 125-Hz vibration: effect of location of contact with vibration. Int Arch Occup Environ Health. 2016 April;89(3):425-33.

11. Ye Y, Griffin M J. Relation between vibrotactile perception thresholds and reductions in finger blood flow induced by vibration of the hand at frequencies in the range 8-250 Hz. Eur J Appl Physiol. 2014 August;114(8):1591-603.

12. Ye Y, Mauro M, Bovenzi M, Griffin M J. Association between vasoconstriction during and following exposure to hand-transmitted vibration. Int Arch Occup Environ Health. 2014 January;87(1):41-9.

13. Ye Y, Griffin M J. Reductions in finger blood flow induced by 125-Hz vibration: effect of area of contact with vibration. Eur J Appl Physiol. 2013 April;113(4):1017-26.

14. Griffin M J. Frequency-dependence of psychophysical and physiological responses to hand-transmitted vibration. Ind Health. 2012;50(5):354-69.

15. Egan C E I, Espie B H, McGrann S, McKenna K M, Allen J A. Acute effects of vibration on peripheral blood flow in healthy subjects. Occup Environ Med. 1996 October;53(10):663-9.

What is claimed is:

1. A process for measuring cutaneous autonomic nerve function which comprises, at least the steps of:
emplacement of a patient contact member having embedded sensors upon the skin of a patient;
activation of the sensors within the patient contact member;
illumination of the skin using Light Emitting Diodes (LEDs) or similar means;
measurement of light reflected back from said skin; and
processing garnered data regarding changes in blood data, whereby a displayed result in generated, and the information is housed in a memory means for the housing of data.

2. The process of claim 1 further comprising:
said processing step including generating a baseline scan, vibratory stimulation reading and a post vibration scan result.

3. The process of claim 1, further comprising:
resultory data housed in a device, which device is operatively and wirelessly linked to a database and cloud means, encrypted secured and otherwise compliant with rules and laws of patient privacy.

4. A product, embodying a device to perform the process of claim 3, having a display
prominently indicating the percentage of blood volume reduction compared to a baseline.

5. The product of claim 4, further comprising:
At least a printed circuit board with control buttons.

6. The product of claim 4, further comprising:
At least a vibration source effective to provide vibrations above 100 Hz.

7. The product of claim 4, further comprising:
At least a vibration source effective to provide vibrations between at least about 120 and 130 Hz.

8. A system for detecting autonomic vasomotor responses to vibrational stimulus comprising, in combination:
At least a vibration source and photoplethysmographic (PPG) sensor controlled through electronic means wherein comparative analysis of waveform data acquired from baseline and post-stimulus measurements is automatically displayed on an interface for a user.

9. The system as defined within claim 8, wherein the at least a vibrational source emits vibrations in the frequency range of from at least about 120 to approximately 130 Hz, and the like types ranges for different applications.

10. The system of claim 9, wherein said vibrational source emits vibrations of constant amplitude.

11. The system of claim 9, wherein said vibrational source emits vibrations which are not of constant amplitude.

12. The system of claim 9, wherein the light emitting diodes within the PPG sensor emit light in the range of at least about 400 to approximately 900 nm.

13. The system of claim 10, wherein the light emitting diodes within the PPG sensor emit light in the range of at least about 400 to approximately 900 nm.

14. The system of claim 12, whereby Pacinian Receptors signal small fiber autonomic nerves controlling skin-based vascular systems along with complementary and supplementary mechanisms related thereto and/or triggered thereby.

15. The system of claim 13, whereby Pacinian Receptors signal small fiber autonomic nerves controlling skin-based vascular systems along with complementary and supplementary mechanisms related thereto and/or triggered thereby.

16. A specialized device housing a system for detecting autonomic vasomotor responses to vibrational stimulus comprising, in combination:
At least a vibration source and photoplethysmographic (PPG) sensor controlled through electronic means wherein comparative analysis of waveform data acquired from baseline and post-stimulus measurements is automatically displayed on an interface for a user.

17. Artificially Intelligent Homunculi operatively linked functionally within subject sensors whereby data arrayed within the subject system are manifested in interfaces for users, stored for further processing and made available strictly on the nature of medical need for patients without impacting privacy concerns.

18. The device of claim 16, whereby the device is disposable.

19. The device of claim 16 whereby the device contains disposable parts exchangeable modularly.

20. The device of claim 19 and applications to link and drive the same with smart-phones, ipad®s and the like personal and group communication devices.
